SEQUENCE LISTING (78 sequences)

SEQ ID NO: 1 - protein, 79 aa, Homo sapiens
SEAEDASLLSFMQGYMKHATKTAKDALSSVQESQVAQQARGWVTDGFSSLKDYWSTVKDKFSEFWDLDPEVRPTSAVAA

SEQ ID NO: 2 - protein, 24 aa, Homo sapiens
MQGYMKHATKTAKDALSSVQESQV

SEQ ID NO: 3 - protein, 10 aa, Homo sapiens
MQGYMKHATK

SEQ ID NO: 4 - protein, 32 aa, Homo sapiens
DGFSSLKDYWSTVKDKFSEFWDLDPEVRPTSA

SEQ ID NO: 5 - protein, 21 aa, Homo sapiens
DGFSSLKDYWSTVKDKFSEFW

SEQ ID NO: 6 - protein, 8 aa, Homo sapiens
MQGYMKHA

SEQ ID NO: 7 - protein, 8 aa, Homo sapiens
QGYMKHAT

SEQ ID NO: 8 - protein, 8 aa, Homo sapiens
GYMKHATK

SEQ ID NO: 9 - protein, 8 aa, Homo sapiens
YMKHATKT

SEQ ID NO: 10 - protein, 8 aa, Homo sapiens
MKHATKTA

SEQ ID NO: 11 - protein, 8 aa, Homo sapiens
KHATKTAK

SEQ ID NO: 12 - protein, 8 aa, Homo sapiens
HATKTAKD

SEQ ID NO: 13 - protein, 8 aa, Homo sapiens
ATKTAKDA

SEQ ID NO: 14 - protein, 8 aa, Homo sapiens
TKTAKDAL

SEQ ID NO: 15 - protein, 8 aa, Homo sapiens
KTAKDALS

SEQ ID NO: 16 - protein, 8 aa, Homo sapiens
TAKDALSS

SEQ ID NO: 17 - protein, 8 aa, Homo sapiens
AKDALSSV

SEQ ID NO: 18 - protein, 8 aa, Homo sapiens
KDALSSVQ

SEQ ID NO: 19 - protein, 8 aa, Homo sapiens
DALSSVQE

SEQ ID NO: 20 - protein, 8 aa, Homo sapiens
ALSSVQES

SEQ ID NO: 21 - protein, 8 aa, Homo sapiens
LSSVQESQ

SEQ ID NO: 22 - protein, 8 aa, Homo sapiens
SSVQESQV

SEQ ID NO: 23 - protein, 8 aa, Homo sapiens
DGFSSLKD

SEQ ID NO: 24 - protein, 8 aa, Homo sapiens
GFSSLKDY

SEQ ID NO: 25 - protein, 8 aa, Homo sapiens
FSSLKDYW

SEQ ID NO: 26 - protein, 8 aa, Homo sapiens
SSLKDYWS

SEQ ID NO: 27 - protein, 8 aa, Homo sapiens
SLKDYWST

SEQ ID NO: 28 - protein, 8 aa, Homo sapiens
LKDYWSTV

SEQ ID NO: 29 - protein, 8 aa, Homo sapiens
KDYWSTVK

SEQ ID NO: 30 - protein, 8 aa, Homo sapiens
DYWSTVKD

SEQ ID NO: 31 - protein, 8 aa, Homo sapiens
YWSTVKDK

SEQ ID NO: 32 - protein, 8 aa, Homo sapiens
WSTVKDKF

SEQ ID NO: 33 - protein, 8 aa, Homo sapiens
STVKDKFS

SEQ ID NO: 34 - protein, 8 aa, Homo sapiens
TVKDKFSE

SEQ ID NO: 35 - protein, 8 aa, Homo sapiens
VKDKFSEF

SEQ ID NO: 36 - protein, 8 aa, Homo sapiens
KDKFSEFW

SEQ ID NO: 37 - protein, 8 aa, Homo sapiens
DKFSEFWD

SEQ ID NO: 38 - protein, 8 aa, Homo sapiens
KFSEFWDL

SEQ ID NO: 39 - protein, 8 aa, Homo sapiens
FSEFWDLD

SEQ ID NO: 40 - protein, 8 aa, Homo sapiens
SEFWDLDP

SEQ ID NO: 41 - protein, 8 aa, Homo sapiens
EFWDLDPE

SEQ ID NO: 42 - protein, 8 aa, Homo sapiens
FWDLDPEV

SEQ ID NO: 43 - protein, 8 aa, Homo sapiens
WDLDPEVR

SEQ ID NO: 44 - protein, 8 aa, Homo sapiens
DLDPEVRP

SEQ ID NO: 45 - protein, 8 aa, Homo sapiens
LDPEVRPT

SEQ ID NO: 46 - protein, 8 aa, Homo sapiens
DPEVRPTS

SEQ ID NO: 47 - protein, 8 aa, Homo sapiens
PEVRPTSA

SEQ ID NO: 48 - protein, 17 aa, Homo sapiens
SEAEDASLLSFMQGYMK

SEQ ID NO: 49 - protein, 14 aa, Homo sapiens
FSSYWMHWVRQVPG

SEQ ID NO: 50 - protein, 19 aa, Homo sapiens
SGVSWNGSRTHYADSVKGR

SEQ ID NO: 51 - protein, 12 aa, Homo sapiens
ARLAGDFWSFDY

SEQ ID NO: 52 - protein, 14 aa, Homo sapiens
CSGSRSNIGSNSVH

SEQ ID NO: 53 - protein, 7 aa, Homo sapiens
RNNQRPS

SEQ ID NO: 54 - protein, 12 aa, Homo sapiens
CATWDASLSTWV

SEQ ID NO: 55 - protein, 14 aa, Homo sapiens
FSTYGMHWVRQAPG

SEQ ID NO: 56 - protein, 19 aa, Homo sapiens
SGVSWNGSRTHYVDSVKRR

SEQ ID NO: 57 - protein, 10 aa, Homo sapiens
ARRSARAFDY

SEQ ID NO: 58 - protein, 14 aa, Homo sapiens
CSGSSSNIGSNYVS

SEQ ID NO: 59 - protein, 7 aa, Homo sapiens
GNSNRPS

SEQ ID NO: 60 - protein, 12 aa, Homo sapiens
CAAWDNSLNGWV

SEQ ID NO: 61 - protein, 14 aa, Homo sapiens
FSNYWIHWVRQAPG

SEQ ID NO: 62 - protein, 19 aa, Homo sapiens
SAISGSGGSTYYADSVKGR

SEQ ID NO: 63 - protein, 8 aa, Homo sapiens
ARARGFDY

SEQ ID NO: 64 - protein, 14 aa, Homo sapiens
CSGSSSNIGTSIVN

SEQ ID NO: 65 - protein, 7 aa, Homo sapiens
GNTNRPS

SEQ ID NO: 66 - protein, 12 aa, Homo sapiens
CAAWDDSLNGPV

SEQ ID NO: 67 - protein, 14 aa, Homo sapiens
FSSYAMSWVRQAPG

SEQ ID NO: 68 - protein, 19 aa, Homo sapiens
SAISGSGGSTYYADSVKGR

SEQ ID NO: 69 - protein, 17 aa, Homo sapiens
ARWRCIPGSCYSAWFDR

SEQ ID NO: 70 - protein, 15 aa, Homo sapiens
CTGSSSNIGAGYDVH

SEQ ID NO: 71 - protein, 7 aa, Homo sapiens
SNNQRPP

SEQ ID NO: 72 - protein, 11 aa, Homo sapiens
CSSYAGSNNLV

SEQ ID NO: 73 - protein, 14 aa, Homo sapiens
FSSYEMNWVRQAPG

SEQ ID NO: 74 - protein, 19 aa, Homo sapiens
SGITWNSGSIGYADSVKGR

SEQ ID NO: 75 - protein, 22 aa, Homo sapiens
AREALYYDFWSGYYRAYYGMDV

SEQ ID NO: 76 - protein, 14 aa, Homo sapiens
CSGSSSNIGSNYVY

SEQ ID NO: 77 - protein, 7 aa, Homo sapiens
RNNQRPS

SEQ ID NO: 78 - protein, 12 aa, Homo sapiens
CAAWDDSLNGWV

(19) United States
(12) Patent Application Publication
(10) Pub. No.: US 2004/0185044 A1
(43) Pub. Date: Sep. 23, 2004

(54) Novel composition

(76) Inventors: Patrick Dhaese, Rixensart (BE); Pascal Mettens, Rixensart (BE); Rene Meykens, Rixensart (BE); Philippe Monteyne, Rixensart (BE); Torbjorn Schiott, Lund (SE); Leif Strandberg, Lund (SE)

Correspondence Address: Andrea Lockenour, GlaxoSmithKline, Corporate Intellectual Property-U.S., UW2220, P.O. Box 1539, King of Prussia, PA, 19406-0939, US

(21) Appl. No.: 10/387,955

(22) Filed: Jul. 15, 2003

(30) Foreign Application Priority Data
Aug. 29, 2002 (WO) PCT/EP02/09650

(51) Int. Cl. A61K 39/395; C07K 16/44

(52) U.S. Cl. 424/141.1; 530/388.15

(57) ABSTRACT

The present invention relates to novel therapies and treatments of atherosclerotic diseases. Accordingly there is provided, methods of treating or preventing atherosclerosis by passive vaccination through administration to a patient of a fully human antibody that is capable of binding to the specific fragments of ApoCIII. Specific human monoclonal antibodies and their use in therapy of atherosclerosis is provided. There is further provided the use of the antibodies of the present invention in medicine.

The present invention relates to novel therapies, and prophylactic treatments of dyslipidaemia, such as atherosclerotic diseases. Accordingly there is provided, methods of treating or preventing atherosclerosis by passive vaccination through administration to a patient of antibodies that are capable of binding to specific epitopes of Apolipoprotein C-III (ApoCIII). The antibodies of the present invention are potent in the prevention, or reduction, of atherosclerotic plaque formation over prolonged periods of time, thereby reducing the potential of atheroslerosis leading to coronary or cerebrovascular disease. There is further provided the use of the antibodies of the present invention in medicine.

Preferred epitopes of ApoCIII which consist of the targets for the passive immunotherapy aspects of the present invention, are encompased within the regions between amino acid numbers 12-35 and between amino acid numbers 45-76 (particuarly 45-65) of the mature form of human ApoCIII, although other regions of ApoCIII may also be targeted by the passive immunotherapy of the present invention.

Atherosclerosis is the leading cause of death and disability in the developed world, and is the major cause of coronary and cerebrovascular deaths, with approximately 7.2 and 4.6 million deaths per year worldwide respectively (Atherosclerosis is generally described in Harrison's Principles of Internal Medicine (14^{th} Edition, McGraw Hill, p1345-1352), Berliner, J. et al., 1995, Circulation, 91:2488-2496; Ross, R., 1993; Nature, 362:801). The name in Greek refers to the thickening (sclerosis) of the arterial intima and accumulation of lipid (athere) in lesions.

Although many generalised or systemic risk factors predispose to its development, such as a high cholesterol diet and smoking, this disease may affect different distinct regions of the circulation. For example, atherosclerosis of the coronary arteries commonly causes angina pectoris and myocardial infarction. Whilst, atherosclerosis of the arteries supplying the central nervous system frequently provokes transient cerebral ischemia and strokes. In the peripheral circulation, atherosclerosis can cause intermittent claudication and gangrene and can jeopardise limb viability. Involvement of the splanchnic circulation can cause mesenteric ischemia and bowel infarction. Atherosclerosis can affect the kidney directly (eg causing renal artery stenosis), and in addition, the kidney is a frequent site of atheroembolic disease.

Atherogenesis in humans typically occurs over many years, usually many decades. The slow build up of atherogenic plaques in the lining of the vasculature can lead to chronic clinical expressions through blood flow restriction (such as stable effort-induced angina pectoris or predictable and reproducible intermittent claudication). Alternatively, a much more dramatic acute clinical event, such as a myocardial infarction or cerebrovascular accident can occur after plaque rupture. The way in which atherosclerosis affects an arterial segment also varies, an additional feature of the heterogeneity and complexity of this disease. Atheromas are usually thought of as stenotic lesions, or plaques, which can limit blood flow, however, atherosclerosis can also cause ectasia and development of aneurysmal disease with an increase in lumen caliber. This expression of atherosclerosis frequently occurs in the aorta, creating a predisposition to rupture or dissection rather than to stenosis or occlusion.

The genesis of atherogenic plaques has been studied in depth. In normal human adults, the intimal layer of arteries contains some resident smooth muscle cells embedded in extracellular matrix and is covered with a monolayer of vascular endothelial cells. Initial stages of atherogenesis involve the development of “fatty streaks” in the walls of the blood vessel resulting from accumulation and deposit of lipoproteins in regions of the intimal layer of the artery. Low-density lipoprotein (LDL) particles, rich in cholesterol, is an example of an atherogenic lipoprotein which is capable of deposition in the vessel walls to form such fatty streaks.

Once deposited within the vessel wall, the lipoprotein particles undergo chemical modification, including both oxidation and non-enzymatic glycation. These oxidised and glycated lipoproteins then contribute to many of the subsequent events of lesion development. The chemical modifications attract macrophages within the vessel walls, which internalise the oxidised LDL and become foam cells which initiate lesions called plaques. It is the atherosclerotic plaques which are responsible for the clinical manifestations of atherosclerosis, either they limit blood flow, or allow aneurism, or may even rupture provoking the coronary or cerebrovascular attacks.

The development of atherosclerosis is a long process which may occur over decades, which is initiated by an imbalance between atherogenic and protective lipoproteins. For example, cholesterol associated with high-density lipoproteins or HDL (so called “good” cholesterol) and low-density lipoproteins or LDL (so called “bad” cholesterol) levels in the circulation are thought to be markers of increased probability of atherosclerosis (Harrison's Principles of Internal Medicine (14^{t} Edition, McGraw Hill, p1345-1352)).

Cholesterol, cholesterol esters, triacylglycerols and other lipids are transported in body fluids by a series of lipoproteins classified according to their increasing density: chylomicrons, Very Low, Low, Intermediate and High density lipoproteins (CM, VLDL, LDL, IDL and HDL respectively). These lipoprotein-complexes consist of a core of hydrophobic lipids surrounded by polar lipids and then by a shell of Apolipoproteins. Currently, there are at least twelve types of apolipoproteins known, A-I, A-Il, A-IV, A-V, B, CI, CII, CIII, D, E, H and J. There are at least two functions of these apolipoproteins which are common to all lipoprotein complexes, first they are responsible for the solubilisation of the hydrophobic lipid cores that they carry, and second they are also involved in the regulation of cholesterol lipoprotein uptake by specific cells. The different types of lipoproteins may have different functions, for example LDL (which are rich in cholesterol esters) are thought to be associated with the transport of cholesterol to peripheral tissues for new membrane synthesis.

One of these apolipoproteins, apolipoprotein C-III (ApoCIII), is a 79 amino acid protein produced in the liver and intestine (Brewer et al., J. Biol. Chem. (1974), 249:4975-4984; Protter, A. A., et al., 1984, DNA, 3:449-456; Fruchart, J. C. et al, 1996, Drugs Affecting Lipid Metabolism, (Eds. Gotto, A. M. et al.), Kluwer Academic Publishers and Fordazione Giovanni Lorenzini, Netherlands, p631-638; Claveny, V. et al., Arteriosclerosis, Thrombosis and Vascular Biology, 15, 7, 963-971; U.S. Pat. No. 4,801,531; McConathy, W. J. et al. 1992, Journal of Lipid Research, 33, 995-1003). ApoCIII is a component of CM, VLDL, LDL (Lenich et al., C., J. Lip. Res. (1988) 29, 755-764), and also HDL, and exists as three isoforms: ApoCIII0, ApoCIII1 and ApoCIII2. ApoCIII0 is not glycosylated, however ApoCHIII1 and ApoCIII2 are glycosylated and have respectively one and two sialic acid residues (Ito et al., 1989 J. lipd. Res. Nov 30:11 1781-1787). The sugar moiety consists of disaccharide β-D galactosyl (1-3) α-N-Acetyl-D-Galactosamine attached to threonine 74 of protein chain by O-glycosidic binding (Assman et al., 1989, BBA 541:234-240). In human normolipidemic plasma ApoCIII0, ApoCIII1 and ApoCIII2 represent 14%, 59% and 27% of total ApoCIII respectively. Mutagenesis of the glycosylation site of human ApoCIII does not affect its secretion and lipid binding (Roghani et al., 1988 JBC 34:17925-32).

Mature Human ApoCIII has the following amino acid sequence:

| | |
|---|---|
| ₁SEAEDASLLSFMQGYMKHATKTAKDALSSVQESQVAQQARGWVTDGFSSL | (SEQ ID.NO. 1) |
| KDYWSTVKDKFSEFWDLDPEVRPTSAVAA₇₉. | |

Plasma concentration of ApoCIII is positively correlated with levels of plasma triglycerides (Schonfeld et al., Metabolism (1979) 28:1001-1010; Kaslyap et al., J. Lip. Res. (1981) 22:800-810). Liver perfusion studies demonstrate that ApoCIII inhibits the hepatic uptake of triglyceride-rich lipoproteins (TRL) and their remnants (Shelburne et al., J. Clin. Inves., (1980) 65:652-658, Windler et al., J. Lip. Res. (1985) 26:556-563). Also in vitro experiments show that ApoCIII inhibit the activity of both lipoprotein lipase (LPL) and hepatic lipase (Brown and Bakinsky, Biochim. Biophs. Acta. (1972) 46: 375-382; Krauss et al., Circ. Res. (1973) 33:403-411; Wang et al., J. Clin. Inves. (1985) 75:384-390; Mc Conathy et al., J. Lip. Res. (1972) 33:995-1003; Kinnemen and Enholm, FEBS (1976) 65:354-357). Moreover, ApoCIII is said to be involved in inhibition of LDL binding to LDL receptors (Fruchart et al. supra), via ApoB.

The role of ApoCIII in plasma TRL metabolism has been more defined by the results of recent studies in transgenic animals (Aalto-Setälä et al., J. Clin. Invest. (1992) 90:5 1889-1900.). Plasma accumulation of TRL in mice overexpressing ApoCIII has been shown to be associated with reduced plasma VLDL and chylomicron clearance (Harrold et al., J. Lip. Res. (1996) 37:754-760) also the inhibitory effect of C apolipoproteins on the LDL receptor of apo B-containing lipoproteins was demonstrated (Clavey et al., Arth. Thromb. and Vasc. Biol. (1995) 15:963-971).

Previously, vaccines in the field of immunotherapy of atherosclerosis have focused on the use of cholesterol as an immunogen to reduce serum cholesterol levels (Bailey, J. M. et al., 1994, Biochemical Society Transactions, 22, 433S; Alving, C. and Swartz, G. M., 1991, Crit. Rev. hmmunol., 10, 441-453; Alving, C. and Wassef, N. M., 1999, Immunology Today, 20, 8, 362-366). Others have attempted to alter the activity of the Cholesterol Ester Transfer Protein (CETP) by vaccination (WO 99/15655). Alternatively, some authors have described vaccines using oxidised LDL as the immunogen, in order to inhibit plaque formation after balloon injury in hypercholesterolemic rabbits (Nilsson, J. et al., 1997, JACC, 30, 7, 1886-1891).

It has been found, surprisingly, that atherosclerosis may be prevented or ameliorated by passive immunotherapy, by reducing or blocking the function of ApoCIII. In particular, the passive immunotherapies of the present invention can be advantageously carried out using specific human antibodies which target epitopes of ApoCIII. The use of the specific antibodies against ApoCIII can focus the immune response to parts of the human ApoCIII molecule without triggering a general response to the whole molecule. Without wishing to be bound by theory, this can be used as a means of distinguishing parts of ApoCIII that are surface exposed on LDL and not HDL, thus focusing the immune response against carriers of “bad cholesterol”, whilst not affecting the positive role of ApoCIII in HDL.

It will be appreciated that ApoCIII can exist in different physiological forms, for example, oxidised and non-oxidised forms, and that allelic variants and mutants of ApoCIII may exist. The antibodies of the present invention may recognise any of these forms of ApoCIII. It is a preferred embodiment that the antibodies recognise ApoCIII having the sequence of SEQ ID No 1, or comprising one or more of the sequences as set out in any of SEQ ID Nos 2-48. In particular, these antibodies will recognise the non-oxidised form of ApoCIII.

The passive immunotherapies of the present invention target an epitope found within the region between amino acid number 1 and 79, or more preferably an epitope found within the region between amino acid number 1 and 17, 12 and 35, or an epitope found within the region between amino acids 45 and 76 of the human ApoCIII molecule as it exists in the circulation of a human, in addition it is preferred that the immunotherapy targets the epitope that is found within the region between amino acid 12 to 21 or 45 to 65 of human ApoCIII.

The sequence of the region between amino acid number 12 and 35 of the human ApoCIII is as follows:

| | |
|---|---|
| MQGYMKHATKTAKDALSSVQESQV. | (SEQ ID NO.2) |

The sequence of the region between amino acid number 12 and 21 of the human ApoCIII is as follows:

| | |
|---|---|
| MQGYMKHATK | (SEQ ID NO.3) |

The sequence of the region between amino acid number 45 and 76 of the human ApoCIII is as follows:

| | |
|---|---|
| DGFSSLKDYWSTVKDKFSEFWDLDPEVRPTSA | (SEQ ID NO: 4) |

The sequence of the region between amino acid number 45 and 65 of the human ApoCIII is as follows:

| | |
|---|---|
| DGFSSLKDYWSTVKDKFSEFW | (SEQ ID NO: 5) |

The present invention also provides the following fragments of the above peptides within which contain an epitope of ApoCIII which may be targeted by the passive immunotherapies of the present invention:

| | |
|---|---|
| Peptide Sequence | SEQ ID NO: |

| | |
|---|---|
| MQGYMKHA | 6 |
| QGYMKHAT | 7 |
| GYMKHATK | 8 |
| YMKHATKT | 9 |
| MKHATKTA | 10 |
| KHATKTAK | 11 |
| HATKTAKD | 12 |
| ATKTAKDA | 13 |
| TKTAKDAL | 14 |
| KTAKDALS | 15 |
| TAKDALSS | 16 |
| AKDALSSV | 17 |
| KDALSSVQ | 18 |
| DALSSVQE | 19 |
| ALSSVQES | 20 |
| LSSVQESQ | 21 |
| SSVQESQV | 22 |
| DGFSSLKD | 23 |
| GFSSLKDY | 24 |
| FSSLKDYW | 25 |
| SSLKDYWS | 26 |
| SLKDYWST | 27 |
| LKDYWSTV | 28 |
| KDYWSTVK | 29 |
| DYWSTVKD | 30 |
| YWSTVKDK | 31 |
| WSTVKDKF | 32 |
| STVKDKFS | 33 |
| TVKDKFSE | 34 |
| VKDKFSEF | 35 |
| KDKFSEFW | 36 |
| DKFSEFWD | 37 |
| KFSEFWDL | 38 |
| FSEFWDLD | 39 |
| SEFWDLDP | 40 |
| EFWDLDPE | 41 |
| FWDLDPEV | 42 |
| WDLDPEVR | 43 |
| DLDPEVRP | 44 |
| LDPEVRPT | 45 |
| DPEVRPTS | 46 |
| PEVRPTSA | 47 |

The sequence of the region between amino acid number 1 and 17 of the human ApoCIII is as follows:

| | |
|---|---|
| ₁SEAEDASLLSFMQGYMK₁₇ | (SEQ ID NO: 48) |

The present invention provides antibodies effective in the prophylaxis or therapy of dyslipidaemia or atherosclerosis which target the epitopes listed in SEQ ID NOs. 1-48, of ApoCIII, and also provides for methods of treatment of atherosclerosis by passive administration of the antibodies of the present invention to individuals in need thereof. Most preferably the antibodies of the invention recognise the epitopes listed in SEQ ID NO: 1, 3, 6-22.

Most preferably the antibodies of the present invention are functional in the treatment of atherosclerosis, and in a preferred form of the present invention they abrogate the inhibition exerted by ApoCIII on the binding of ApoB to its receptor, and/or the activity of lipoprotein lipase. Such activities may readily be assayed by the man skilled in the art for example by methods described in Fruchard et al, supra; and McConathy et al., supra.

The antibodies of the present invention are provided for use in medicine, and for use in the treatment or prevention of atherosclerosis.

The antibodies of the present invention will be generally administered for both initial and boosting doses. It is expected that the boosting doses will be adequately spaced, or preferably given at such times where the levels of circulating antibody fall below a desired level.

The antibody preparations of the present invention may be used to protect or treat a mammal susceptible to, or suffering from atherosclerosis, by means of administering said antibodies via a systemic route. These administrations may include injection via the intramuscular, intraperitoneal, intradermal or subcutaneous routes. The antibodies will preferably be administered in a pharmaceutical composition, together with a pharmaceutically acceptable carrier.

In one aspect of the present invention are provided fully human monoclonal antibodies capable of binding to epitopes of SEQ ID NOs: 1 to 48 (preferably SEQ ID NO: 1, 2 or 3) in the context of the human ApoCIII molecule, and their use in immunotherapy.

Antibodies typically comprise two heavy chains linked together by disulphide bonds and two light chains. Each light chain is linked to a respective heavy chain by disulphide bonds. Each heavy chain has at one end a variable domain followed by a number of constant domains. Each light chain has a variable region at one end and a constant domain at its other end. The light chain variable domain is aligned with the variable domain of the heavy chain. The light chain constant domain is aligned with the first constant domain of the heavy chain.

The constant domains in the light and heavy chains are not involved directly in binding the antibody to antigen. The variable domains in each pair of light and heavy chains form the antigen binding site. The domains on the light and heavy chains have the same general structure and each domain comprises a framework of four regions, whose sequences are relatively conserved, connected by three complementarity determining regions (CDRs). The four framework regions largely adopt a beta sheet conformation and the CDRs form loops connecting, and in some cases forming part of, the beta-sheet structure. The CDRs are held in close proximity by the framework regions and, with the CDRs from the other domain, contribute to the formation of the antibody binding site.

The preparation of altered antibodies in which the variable region of a rodent antibody is combined with the constant region of a human antibody is now well known in the the art (Oi and Morrison 1986 Biotechniques 4, 214-212). Humanised antibodies in which the CDRs are derived from a source different from that of the framework of the antibody's variable domains are disclosed in EP-A-0239400. The CDRs may be derived from a rodent or primate monoclonal antibody, or by screening a human phage-display library using known techniques (eg WO01/75091, WO98/32845). If the CDRs are obtained from a non-human source, and are introduced into the scaffold of a human antibody, such hybrid antibodies are known “humanised” antibodies. If the CDR regions are obtained by use of a human phage display library, and are introduced into the scaffold of a human antibody, then the resulting antibodies are fully human. Preferably, antibodies of the present invention are fully human antibodies. The antibodies should not be recognisable by the host human immune system as foreign, or should elicit a far reduced immune response when administered to a human than the immune response mounted by a human against a humanised or a chimaeric antibody which has regions deived from eg a rodent or primate.

The CDR sequences of preferred human antibodies of the present invention are shown in SEQ ID Nos: 49 to 78 in Table 1 on the following page. Most preferred antibodies are those having a CDR as shown in SEQ ID Nos: 49 to 66.

TABLE 1

| | | | |
|---|---|---|---|
| Name | H1 | H2 | H3 |

ATH1C23 Completed sequences

| | | | |
|---|---|---|---|
| ATH1C23-4.MH | FSSYWMHWVRQVPG | SGVSWNGSRTHYADSVKGR | ARLAGDFWSFDY |
| | (SEQ ID NO 49) | (SEQ ID NO 50) | (SEQ ID NO 51) |
| ATH1C23-21.MH | FSTYGMHWVRQAPG | SGVSWNGSRTHYVDSVKRR | ARRSARAFDY |
| | (SEQ ID NO 55) | (SEQ ID NO 56) | (SEQ ID NO 57) |

ATH3 Completed sequences

| | | | |
|---|---|---|---|
| ATH32.MH | FSNYWIHWVRQAPG | SAISGSGGSTYYADSVKGR | ARARGFDY |
| | (SEQ ID NO 61) | (SEQ ID NO 62) | (SEQ ID NO 63) |
| ATH34.MH | FSSYAMSWVRQAPG | SAISGSGGSTYYADSVKGR | ARWRCIPGSCYSAWFDR |
| | (SEQ ID NO 67) | (SEQ ID NO 68) | (SEQ ID NO 69) |

ATH1C3 Completed sequences

| | | | |
|---|---|---|---|
| ATH1C3-1.MH | FSSYEMNWVRQAPG | SGITWNSGSIGYADSVKGR | AREALYYDFWSGYYRAYYGMDV |
| | (SEQ ID NO 73) | (SEQ ID NO 74) | (SEQ ID NO 75) |

| | | | |
|---|---|---|---|
| Name | L1 | L2 | L3 |

ATH1C23 Completed sequences

| | | | |
|---|---|---|---|
| ATH1C23-4.MH | CSGSRSNIGSNSVH | RNNQRPS | CATWDASLSTWV |
| | (SEQ ID NO 52) | (SEQ ID NO 53) | (SEQ ID NO 54) |
| ATH1C23-21.MH | CSGSSSNIGSNYVS | GNSNRPS | CAAWDNSLNGWV |
| | (SEQ ID NO 58) | (SEQ ID NO 59) | (SEQ ID NO 60) |

ATH3 Completed sequences

| | | | |
|---|---|---|---|
| ATH32.MH | CSGSSSNIGTSIVN | GNTNRPS | CAAWDDSLNGPV |
| | (SEQ ID NO 64) | (SEQ ID NO 65) | (SEQ ID NO 66) |
| ATH34.MH | CTGSSSNIGAGYDVH | SNNQRPP | CSSYAGSNNLV |
| | (SEQ ID NO 70) | (SEQ ID NO 71) | (SEQ ID NO 72) |

ATH1C3 Completed sequences

| | | | |
|---|---|---|---|
| ATH1C3-1.MH | CSGSSSNIGSNYVY | RNNQRPS | CAAWDDSLNGWV |
| | (SEQ ID NO 76) | (SEQ ID NO 77) | (SEQ ID NO 78) |

A fully human monoclonal antibody that recognises the region 12-35 of human ApoCIII is ATH1C3-1. Fully human monoclonal antibodies that recognise the region 45-65 of human ApoCIII are ATH3-2 and ATH3-4. Fully human monoclonal antibodies that recognise the region 1-76 of human ApoCIII are the antibodies mentioned above, together with: ATH1C23-4 and ATH1C23-21, which recognise epitopes which do not fall within the sequences 12-35 and 45-65.

The sequences of the hypervariable regions and the complementarity determining regions (CDRs) of the fully human antibodies of the present invention are fully encompassed within the present invention.

Also encompassed within the scope of the present invention are “similar” human antibodies to the above identified monoclonal antibodies. For example, the present invention also provides other antibodies that have a similar amino acid sequence in its hypervariable regions, and/or similar CDR, so that the antibody is capable of competing with the fully human antibody for binding to ApoCIII. Thus, in a competition assay at least 40%, preferably at least 50, 60, 70, 80 or 90%, of the “similar” antibodies will bind to ApoCIII in the presence of antibodies with non-modified CDRs. Suitably, the CDRs of an antibody according to the invention are the light chain CDRs L1 to L3 and the heavy chain CDRs H1 to H3 as described in any of the SEQ ID Nos 49 to 78.

The amino acid sequences of these CDRs may be modified, however. The amino acid sequence of each CDR may be modified by amino acid substitutions, insertions and/or deletions as described below.

Each CDR may therefore include one or two amino acid substitutions, insertions and/or deletions. There may be up to three amino acid substitutions, insertions and/or deletions in light chain CDRL3 or heavy chain CDRH3. Up to four amino acid substitutions, insertions and/or deletions may be present in light chain CDRL1. Up to six amino acid substitutions, insertions and/or deletions may be present in heavy chain CDRH2. Preferably the amino acid sequence of each CDR is substantially homologous to that of each CDR set out above.

Preferably the degree of sequence identity is at least 50% and more preferably it is at least 75%. Sequence identities of at least 90, 91, 92, 93, 94, 95, 96, 97, 98 or 99% are most preferred.

It will nevertheless be appreciated by the skilled person that high degrees of sequence identity are not necessarily required since various amino acids may often be substituted for other amino acids which have similar properties without substantially altering or adversely affecting certain properties of a protein. These are sometimes referred to as “conservative” amino acid changes. Thus the amino acids glycine, valine, leucine or isoleucine can often be substituted for one another. Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains) and cysteine and methionine (amino acids having sulphur containing side chains). Thus the term “derivative” can also include a variant of an amino acid sequence comprising one or more such “conservative” changes relative to said sequence.

The term “antibody” herein is used to refer to a molecule having a useful antigen binding specificity, ie will recognise and bind to ApoCIII. Those skilled in the art will readily appreciate that this term may also cover polypeptides which are fragments of or derivatives of antibodies yet which can show the same or a closely similar functionality. Such antibody fragments or derivatives are intended to be encompassed by the term antibody as used herein.

The term “monoclonal antibody” is used herein to encompass any isolated antibodies such as conventional monoclonal antibody hybridomas, but also to encompass isolated monospecific antibodies produced by any cell, such as for example a sample of identical human immunoglobulins expressed in a mammalian cell line.

The monoclonal antibodies of the present invention are capable of being used in passive prophylaxis or therapy, by administration of the antibodies into a patient, for the amelioration of atherogenic disease.

The monoclonal antibodies of the present invention may be generated by screening a phage-display library to produce a scFv sequence which corresponds to a CDR and producing fully human IgG antibodies having this CDR region using known techniques, eg. as described in WO01/75091.

Hybridomas secreting the monoclonal antibodies of the present invention are also provided. Preferably, these hybridomas are of eukaryotic or insect origin. Most preferably, the hybridomas are eukaryotic, in order to reduce the presence of endotoxin and also to provide for appropriate eukaryotic processing of the antibodies.

Pharmaceutical compositions comprising the antibodies, described above, also form an aspect of the present invention. Also provided are the use of the antibodies in medicine, and in the manufacture of medicaments for the treatment of atherosclerosis.

In the passive treatments of atherosclerosis as provided herein, the administration of the antibodies of the present invention will be administered (preferably intra-venously) to the patients in need thereof. The frequency of administration may be determined clinically by following the decline of antibody titres in the serum of patients over time, but in any event may be at a frequency of 1 to 52 times per year, and most preferably between 1 and 12 times per year. Quantities of antibody may vary according to the severity of the disease, or half-life of the antibody in the serum, but preferably will be in the range of 1 to 10 mg/kg of patient, and preferably within the range of 1 to 5 mg/kg of patient, and most preferably 1 to 2 mg/kg of patient.

The immunogens, immunogenic compositions, vaccines or monoclonal antibodies of the present invention may be administered to a patient who is suffering from, or is at risk to, atherosclerotic disease, and are effective in re-establishing the correct equilibrium of the “bad” lipoproteins (apo B containing lipoproteins) to the “good” lipoproteins (apo A-I containing lipoproteins) balance, and minimise the circulation time of apo B containing lipoproteins. Not wishing to be bound by theory, the inventors believe that these functions minimise the possibility of deposit and oxidation of apo B containing lipoproteins within the blood vessel walls, and hence, reduce the risk of atherosclerotic plaque formation or growth. Preferably, the antibodies are administered to a patient who is considered to be at high risk of developing atherosclerotic disease, at an early time point before disease is fully, or partially, established.

The present invention, therefore, provides the use of the anti-ApoCIII monoclonal antibodies of the present invention, as defined above, in the manufacture of pharmaceutical compositions for the prophylaxis or therapy of atherosclerosis. Accordingly, the anti-ApoCIII monoclonal antibodies of the present invention are provided for use in medicine, and in the medical treatment or prophylaxis of atherosclerosis.

There is also provided a method of treatment or prophylaxis of atherosclerosis comprising the administration to a patient suffering from or susceptible to atherosclerosis, of an antibody of the present invention.

A method of prophylaxis or treatment of atherosclerosis is provided which comprises a reduction of total circulating triglyceride levels in a patient, by the administration of an antibody of the present invention to the patient. In particular there is provided a method of reducing the amount of circulating VLDL and LDL in a patient, by the administration of the antibodies of the present invention to the patient.

Also provided is a method of prophylaxis or treatment of atherosclerosis by the administration to a patient of an antibody which is capable of reducing the average circulation time of ApoB containing lipoproteins. In this regard the average circulation time of ApoB containing lipoproteins, may be investigated in an in vivo animal model by the measuring the clearance rate of labelled ApoB containing lipoproteins from the plasma of the mammal (half-life of labelled ApoB containing lipoproteins).

A preferred antibody for these method of treatment aspects of the present invention recognises any one of the ApoCIII epitopes SEQ ID NO: 1-48. A particularly preferred antibody has a CDR of any of SEQ ID No 49 to 78, or a modification thereof as described herein. Particularly preferred antibodies have CDRs 49 to 54, 55 to 60, 61 to 66, 67 to 72, or 73 to 78, as shown for ATH1C23-4.MH, ATH1C23-21.MH, ATH32.MH, ATH34.MH, ATH1C3-1.MH, in Table 1.

In addition, human antibodies which recognise any ApoCIII molecule, or fragments, mutants, homologues, analogues or chemically or biologically modified versions thereof are also included.

Surprisingly, targetting of ApoCIII may be used to downregulate the negative effects of the “bad” cholesterol (LDL), whilst not having a negative effect on the “good” cholesterol (HDL).

Preferred antibody isotypes of the present invention are IgG1 and IgG4. IgG4 isotypes are particularly preferred because they are thought to have lower affinity for Fcγreceptors and thus be less efficient in mediating antibody-dependent complement-mediated cytolysis (ADCC; Adair Immunol Rev 1992;5-39.)

Preferred methods of treating individuals suffering from Atherosclerosis having elevated levels of circulating ApoCIII in their plasma comprise reducing the levels of circulating ApoCIII, by the administration of a monoclonal Ab that is capable of blocking the activity of ApoCIII, by binding to the epitope of any of SEQ ID NO: 1-48 and thereby abrogating the ApoCIII-mediated inhibition of lipoprotein lipase and/or the binding of ApoB to its receptor, to said patient.

Also provided by the present invention is a method of treatment or prophylaxis of atherosclerosis by reducing the number of ApoCIII molecules which are associated with an ApoB molecule in situ in the context of a lipoprotein by administration of a monoclonal antibody of the present invention. In a normal individual there is approximately one ApoB present in an LDL particle, the ApoB being associated with between 1-5 ApoCIII molecules. In diseased individuals the number of ApoCIII molecules may increase to up to 25. Accordingly, there is provided by the present invention a method of treatment or prophylaxis of atherosclerosis by reducing the ratio of ApoCIII molecules per ApoB molecules in the LDL in an individual with atherosclerosis from a high disease state level (approximately 20 to 25:1) to a reduced therapeutic level preferably below 15:1, more preferably below 10:1 and more preferably below 5:1, preferably below 3:1, and most preferably approximately 1:1 ApoC:ApoB. Levels of ApoCIII contained within ApoB-containing lipoproteins may be measured by nephelometry or electro-immunodiffusion (normal range is 2 to 3 mg/dL).

Also provided by the present invention is a combination therapy for treatment or prophylaxis of atherosclerosis comprising the passive immunotherapy of the present invention in combination with any other therapy or combination of therapies for treatment or prophylaxis of atherosclerosis, such as immunotherapy directed towards modified ApoCIII, oxidised ApoA, oxidised ApoB (as described in WO02/080954), oxidised LDL (WO02/50550) or cholesterol ester transfer protein (CETP; WO99/15655), or other known therapies.

The present invention is illustrated, but not limited, by the following examples:

### EXAMPLES

### Example 1

### Peptide Synthesis

The ApoCIII peptides (1-79, 12-35 and 45-65) were synthesised by the solid phase method (Merrifield, 1986) on an automated synthesiser Model ABI 433A (Applied Biosystems Inc.) using Boc/Bzl strategy on a Boc-Ala-PAM resin for total ApoCIII and MBHA resin for the others fragments. Other ApoCIII peptides may be synthesised according to the same method. Each amino acid was coupled twice by dicyclohexylcarbodiimide/hydroxybenzotriazole without capping. Side chain protecting groups were as follows: Arg(Ts), Asp(Ochex), Glu(Ochex), Lys(2-Cl-Z), His(Dnp), Ser(Bzl), Thr(Bzl), Met(O) and Tyr(Br-Z). According to the sequence, the group Dnp on His was removed from the peptide, prior to the cleavage from its support by treatment with 10% β-mercaptoethanol, 5% diisopropylethylamine in DCM for 2 h and in NMP for 2 h. The peptidyl resin was then treated with 50% TFA in DCM for 20 min to remove the amino-terminal Boc. The peptide was cleaved from the resin and simultaneously deprotected according to a low and high HF procedure: the resin (1 g) was treated with anhydrous HF (2.5 mL) in the presence of p-cresol (0.75 g), p-thiocresol (0.25 g) and dimethylsulfide (6.5 mL) at 0° C. After 3 h hydrogen fluoride and dimethylsulfide were removed by vacuum evaporation and the residual scavengers and by products were extracted with diethyl ether. The reaction vessel was recharged with p-cresol (0.75 g), p-thiocresol (0.25 g) and 10 ml of anhydrous HF and the mixture was allowed to react at 0° C. for 1.5 h. Hydrogen fluoride was removed by evaporation and the residue was triturated with diethyl ether. The residue was filtered off, washed with diethyl ether and extracted with 200 ml of 10% aqueous acetic acid and lyophilised. The crude product was analysed by reversed-phase HPLC on a Vydac C18 column (4,6×250 mm, 5μ, 100 A) using 60 min linear gradient from 0 to 100% Buffer B (Buffer A: 0.05% TFA in H₂O and Buffer B: 0.05% TFA, 60% CH₃CN in H₂O) at flow rate of 0.7 ml/min and detection was performed at 215 nm. Synthetic peptides were purified by RP-HPLC and were characterised and analysed by HPLC, the molecular mass determined by spectrometry.

### Example 2

### Monoclonal Antibody Production

Methods of production of recombinant antibodies, by screening phage display libraries, such as scFv or Fab libraries, are well known in the art (McCafferty et al 1990 Nature 348, 552-554; Barbas et al 1991 Proc. Natl. Acad. Sci. USA 88, 7978-7982; Clarkson et al 191, Nature 352, 624-628; Söderlind et al 2000 Nature Biotech. 8, 852-856). The framework regions of antibodies derived from the n-CoDeR™ scFv library used in the present example are shown below. CDR regions (H1-H3 and L1-L3) are indicated.

Heavy chain region
EVQLLESGGGLVQPGGSLRLSCAASGFT--- H1 ---KGLEWV--- H2 ---
FTISRDN
SKNTLYLQMNSLRAEDTAVYYC--- H3 ---WGQGTLVTVSS
Light chain region
QSVLTQPPSASGTPGQRVTIS--- L1 ---WYQQLPGTAPKLLIY--- L2 ---GVP
DRFSGSKSGTSASLAISGLRSEDEADYY--- L3 ---FGGGTKLTVLG

Peptides synthesised as described in Example 1 may be used to select and screen e.g. the n-CoDeR™ scFv library to identify scFv fragments, which are capable of binding to the peptides. Selection of scFv or Fab phage display libraries can be performed in many different ways, employing various techniques to extract target binding phages, e.g immobilisation of target antigens to solid surfaces or capture of biotinylated target antigens on streptavidin coated magnetic beads such as Dynabeads (Dynal, Norway)

Briefly, peptides (1-79), (12-35) and (45-65) were used to perform three consecutive selections against the target peptides of Example 1, as described below

Selection 1: For each of the 3 target peptides approximately 10¹³ n-CoDeR™ phages in PBS containing 3% BSA, 0.05% Tween 20 and 0.02% sodium azide, were incubated for 1 h with 1×10⁻⁷ M of the 3 different biotinylated target peptides in 1.8 ml. Biotinylated target antigens were then captured on streptavidin coated magnetic Dynabeads. Non-specific phages were removed by washing with the buffer described above. Bound phages were eluted with trypsin digestion and used to infect Escherichia coli HB101F′ for phage amplification before selection 2.

Selection 2: The amplified phage pool from selection on peptide(1-79) was divided into two parts and used for selection on

a) 2×10⁻⁸ M biotinylated peptide (1-79) in 1 ml buffer, as above. In addition, 1×10⁻⁷ M of non-biotinylated peptide (12-35) and peptide (45-65) were used for counter-selection, thereby increasing the probability to find fragments binding to other regions of peptide (1-79) than regions (12-35) nor (45-65).

b) 2×10⁻⁸ M biotinylated peptide (1-79), as above. In addition, 1×10⁻⁷ M of non-biotinylated peptide (12-35) and peptide(45-65) were used for competition, thereby increasing the probability to find fragments binding to other regions of peptide (1-79) than regions (12-35) nor (45-65).

Amplified phage pools from selection on peptide (45-65), were further selected on the biotinylated using competition with non-biotinylated target peptides.

Selection 3: Selection was performed on non-biotinylated target antigens immobilised in microtiter plate wells, 2 pmole/well. Each selections were performed in 100 μl in 8 microtiter wells per target. The amplified phage pool from selection on peptide (1-79) was divided into three parts and used for selection on

a) peptide (1-79) with counter-selection on 1×10⁻⁶ M biotinylated peptide (12-35) captured on Dynabead and competition by 1×10⁻⁶ M non-biotinylated peptide (12-35).

b) peptide (1-79) with counter-selection on 1×10⁻⁶ M biotinylated peptide (45-65) captured on Dynabead and competition by 1×10⁻⁶ M non-biotinylated peptide (45-65).

c) Peptide (1-79) with counter-selection on 1×10⁻⁶ M biotinylated peptide (12-35 captured on Dynabeads, 1×10⁻⁶ M biotinylated peptide (45-65) captured on Dynabeads, and 1×10⁻⁶ M non-biotinylated peptides (12-35) and (45-65)

Amplified phage pools from selection on peptide (12-35), were selected on peptide (12-35) and counter-selected on Dynabead coupled peptide (45-65) as well as competed non-biotinylated peptide.

Amplified phage pools from selection on peptide (45-65), were selected on peptide (45-65) and counter-selected on Dynabead coupled peptide (12-35) as well as competed non-biotinylated peptide.

Candidate target binding scFv clones were then identified in a screening process briefly described below:

The selected phage pools from selection 3 were then converted to scFv format by enzymatic cleavage of the phagemid, deleting the phage gene III. The resulting plasmids encoding soluble scFv were transformed into E. coli.

Single bacterial colonies were picked for growth in LB-medium, and expression of soluble scFv was induced by addition of IPTG. The resulting scFv stocks were assayed for binding to the biotinylated peptides, loaded in avidin pre-coated wells, in Luminescence ELISA. Positive clones were further assayed in Luminescence ELISA against the target peptides, directly coated to the wells.

Positive clones from all groups were sequenced, and identical scFv clones were excluded. Chosen clones were expressed and purify by e.g protein A affinity chromatography.

correct binding of the purified scFv clones were confirmed in ELISA

The chosen clones may preferably be converted to IgG1 or IgG4 format using techniques well known in the art, for example as described in Henderikx et al., (2002; Am. J. Pathol. 160, 1597-1608).

The clones may be transferred to a IgG1 or IgG4 vector and the functionality of the clones tested by transient expression in, for example, Cos 7 cells.

Functional clones may then be transfected into NSO cells and bulk pools expanded.

Human anti-ApoCIII antibodies
Recognition of HDL, VLDL, native isolated ApoCIII (ApoCIII nat) and synthetic
ApoCIII peptides 1-79, 1-17, 12-21, 12-35, 45-65, 45-76

| | | | | synthetic ApoCIII | | | | | |
|---|---|---|---|---|---|---|---|---|---|
| | VLDL | HDL | ApoCIII nat | 1-79 | 1-17 | 12-21 | 12-35 | 45-65 | 45-76 |
| C23-4 IgG1 | +++ | +++ | +++ | +++ | +++ | − | − | − | − |
| (SEQ ID Nos 49-54) | | | | | | | | | |
| C23-4 IgG4 | +++ | +++ | +++ | +++ | +++ | − | − | − | − |
| (SEQ ID Nos 49-54) | | | | | | | | | |

Specific Response IgG4 > IgG1 (IgG4 ˜ IgG tot)

| | | | | | | | | | |
|---|---|---|---|---|---|---|---|---|---|
| C23-21 IgG1 | +++ | +++ | +++ | +++ | − | − | − | − | +++ |
| (SEQ ID Nos 55-60) | | | | | | | | | |
| C23-21 IgG4 | +++ | +++ | +++ | +++ | − | − | − | − | +++ |
| (SEQ ID Nos 55-60) | | | | | | | | | |

Specific Response IgG4 > IgG1 (=0)

| | | | | | | | | | |
|---|---|---|---|---|---|---|---|---|---|
| C3-1 IgG1 | +++ | +++ | +++ | +++ | + | + | +++ | + | + |
| (SEQ ID Nos 61-66) | | | | | | | | | |
| C3-1 IgG4 | +++ | +++ | +++ | +++ | + | + | +++ | + | + |
| (SEQ ID Nos 61-66) | | | | | | | | | |

Specific Response IgG4 > IgG1 (=0)

| | | | | | | | | | |
|---|---|---|---|---|---|---|---|---|---|
| 3-2 IgG1 | ++ | ++ | ++ | ++ | − | − | − | +/− | − |
| (SEQ ID Nos 67-72) | | | | | | | | | |
| 3-2 IgG4 | +(+) | +(+) | +(+) | +(+) | − | − | − | +/− | − |
| (SEQ ID Nos 67-72) | | | | | | | | | |

Specific Response IgG4 > IgG1 (=0)

| | | | | | | | | | |
|---|---|---|---|---|---|---|---|---|---|
| 3-4 IgG1 | + | +++ | + | +/− | − | − | − | +/− | +/− |
| (SEQ ID Nos 73-78) | | | | | | | | | |
| 3-4 IgG4 | ++ | +++ | ++ | +/− | − | +/− | − | ++ | + |
| (SEQ ID Nos 73-78) | | | | | | | | | |

Specific Response IgG4 > IgG1 (=0)

1. Use of a fully human antibody which recognises ApoCIII, for the manufacture of a medicament for the treatment of atherosclerosis or dyslipidaemia, or diseases associated therewith or resulting therefrom.

2. Use according to claim 1 in which the antibody has a CDR corresponding to any of SEQ ID Nos 49 to 78, or modifications thereof, which recognise human ApoCIII or peptides, fragments or mimotopes thereof.

3. Use according to claim 1 or 2 in which the antibody has a CDR corresponding to any of SEQ ID Nos 49 to 66, or modifications thereof, which recognise human ApoCIII or peptides, fragments or mimotopes thereof.

4. A pharmaceutical composition comprising human monoclonal antibody having a CDR corresponding to any of SEQ ID Nos 49 to 78, or modifications thereof, which recognise human ApoCIII or peptides, fragments or mimotopes thereof, together with a pharmaceutically acceptable excipient.

5. A fully human antibody which recognises ApoCIII, for use in medicine.
